(19) United States
(12) Patent Application Publication
(10) Pub. No.: US 2026/0240777 A1
(43) Pub. Date: Aug. 20, 2026

(54) PHARMACEUTICAL COMPOSITION COMPRISING RUXOLITINIB

(71) Applicants: Zhejiang Kisun Pharmaceutical Co., Ltd., Taizhou (CN); STADA Arzneimittel AG, Bad Vilbel (DE)

(72) Inventors: Jun Liu, Taizhou (CN); Chunguang Dai, Taizhou (CN); Guanneng Yu, Taizhou (CN); Zhiguo Zheng, Taizhou (CN); Jessica Schönborn, Bad Vilbel (DE); Niklas Renner, Oberursel (DE); Andreas Wiederer, Mainstockheim (DE)

(73) Assignees: Zhejiang Kisun Pharmaceutical Co., Ltd., Taizhou (CN); STADA Arzneimittel AG, Bad Vilbel (DE)

(21) Appl. No.: 19/164,324

(22) PCT Filed: Mar. 14, 2024
(86) PCT No.: PCT/EP2024/056774

§ 371 Date: Sep. 11, 2025

(30) Foreign Application Priority Data
Mar. 15, 2023 (WO) PCT/CN2023/081660

(51) Int. Cl. A61K 9/20; A61K 31/519

(52) U.S. Cl. CPC A61K 9/2027; A61K 31/519

(57) ABSTRACT

A solid oral pharmaceutical composition contains ruxolitinib or a pharmaceutically acceptable salt thereof as an active pharmaceutical ingredient. The composition contains less than 2.0% by weight of povidone. Moreover, a process for the preparation of the pharmaceutical composition is disclosed. The pharmaceutically acceptable salt of ruxolitinib may be selected from a ruxolitinib phosphoric acid salt, a ruxolitinib maleic acid salt, a ruxolitinib sulfuric acid salt, and a ruxolitinib oxalic acid salt.

### CROSS REFERENCE TO RELATED APPLICATIONS

This application is a national stage application of international patent application PCT/EP2024/056774, filed Mar. 14, 2024, designating the United States and claiming priority from PCT/CN2023/081660, filed Mar. 15, 2023, and the entire content of both applications is incorporated herein by reference.

### TECHNICAL FIELD

The present invention relates to a solid oral pharmaceutical composition comprising as an active pharmaceutical ingredient ruxolitinib or a pharmaceutically acceptable salt thereof.

### BACKGROUND

Ruxolitinib is the International Nonproprietary Name (INN) for the drug molecule with the chemical name (R)-3-(4-(7H-pyrrolo[2,3-d]pyrimidin-4-yl)-1H-pyrazol-1-yl)-3-cyclopentylpropanenitrile. Ruxolitinib per se, i.e. Ruxolitinib in the form of its free base, has the following chemical structure:

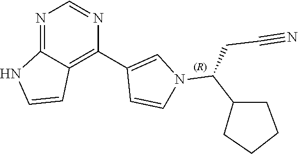

Ruxolitinib is a selective inhibitor of the Janus Associated Kinases (JAKs) JAK1 and JAK2, that mediate the signaling of a number of cytokines and growth factors that are important for hematopoiesis and immune function. Ruxolitinib inhibits the JAK-STAT signaling and cell proliferation of cytokine-dependent cellular models of hematological malignancies, that play a role in regulating the development, proliferation, and activation of several immune cell types important for graft versus host disease pathogenesis.

In Europe ruxolitinib is marketed under the trade name Jakavi®. Jakavi® is indicated for the treatment of myelofibrosis, polycythaemia vera and graft versus host disease.

According to its summary of product characteristics Jakavi® is formulated as a filmless tablet that contains 5 mg, 10 mg, 15 mg or 20 mg ruxolitinib phosphate salt (calculated as ruxolitinib free base) and consists of the further constituents microcrystalline cellulose, magnesium stearate, colloidal anhydrous silica, sodium starch glycolate, povidone, hydroxypropyl cellulose (low substituted) and lactose monohydrate. Investigations revealed that Jakavi® contains 2% by weight of povidone based on the total weight of the Jakavi® formulation.

As already mentioned above Jakavi® contains the drug molecule ruxolitinib in form of a phosphate salt. The Ruxolitinib phosphate salt is disclosed in EP 2 173 752 A2 that discloses also a maleic acid salt and a sulfuric acid salt of ruxolitinib. In EP 2 173 752 A2 it is stated that said ruxolitinib salts have a superior aqueous solubility, rate of dissolution and chemical stability compared with the free base form of ruxolitinib.

However, there is still a need to provide solid oral pharmaceutical compositions comprising ruxolitinib that are chemically stable with respect to ruxolitinib, e.g. in order to provide a relatively long high product quality, a relatively long shelf life of the composition and/or to permit more flexible storage conditions.

### SUMMARY

Thus, the object of the present invention is to provide a solid oral pharmaceutical composition comprising as an active pharmaceutical ingredient ruxolitinib or a pharmaceutically acceptable salt thereof, wherein the composition is relatively stable with respect to the chemical stability of ruxolitinib.

This object has been achieved by a solid oral pharmaceutical composition comprising as an active pharmaceutical ingredient ruxolitinib or a pharmaceutically acceptable salt thereof wherein the composition contains less than 2.0% by weight of povidone calculated with reference to the total weight of the pharmaceutical composition.

Surprisingly it has been found, that the less the povidone-content of the pharmaceutical composition according to the present invention is, the more is the stability of the pharmaceutical composition with respect to the chemical stability of ruxolitinib.

Even it could be shown, that the solid oral pharmaceutical composition according to the invention is be more stable with respect to the chemical stability of ruxolitinib compared to the Jakavi® composition.

Moreover, it has been found that the pharmaceutical composition according to the present invention shows a sufficient solubility and dissolution rate to permit an effective oral bioavailability.

Furthermore, it has been found that the pharmaceutical composition according to the present invention can be formulated in a way that is bioequivalent to the Jakavi® product.

As already said above the solid oral pharmaceutical compositions is relatively stable with respect to the chemical stability of ruxolitinib.

### DESCRIPTION OF EXEMPLARY EMBODIMENTS

The term “stability” in this context is understood to mean the definition worked out by the Arbeitsgemeinschaft für pharmazeutische Verfahrenstechnik (APV), according to which “stability” means the specification-suitable quality of the medicament up to the end of the shelf life fixed by the producer. The quality of the medicament is thus determined by the active ingredient content and the purity, the sensorially perceivable, physical-chemical and the microbiological properties, wherein the active ingredient content should not up to the end of the shelf life fall below 90% of the declared value.

It is preferred that the pharmaceutical composition according to the present invention has at a temperature of 30° C.±2° C. and a relative humidity RH of 65%±5% (see committee for proprietary medicinal products (CPMP), “guideline on stability testing: stability testing of existing active substances and related finished products”, CPMP/QWP/122/02, rev 1 corr of Dec. 17, 2003), a stability or shelf life of at least 1 year, preferably of at least 2 years, more preferably of at least 3 years, more preferably of at least 3.5 years, more preferably of at least 4 years, more preferably of at least 4.5 years and even more preferably of at least 5 years.

Moreover it is preferred that the pharmaceutical composition according to the present invention has at a temperature of 30° C.±2° C. and a relative humidity RH of 65%+5% (see committee for proprietary medicinal products (CPMP), “guideline on stability testing: stability testing of existing active substances and related finished products”, CPMP/QWP/122/02, rev 1 corr of Dec. 17, 2003), a stability or shelf life of up to 1 year, preferably of up to 2 years, more preferably of up to 3 years, more preferably of up to 3.5 years, more preferably of up to 4 years, more preferably of up to 4.5 years and even more preferably of up to 5 years.

As already stated above the present invention relates to a solid oral pharmaceutical composition comprising as an active pharmaceutical ingredient ruxolitinib or a pharmaceutically acceptable salt thereof wherein the composition contains less than 2.0% by weight of povidone calculated with reference to the total weight of the pharmaceutical composition.

According to a preferred embodiment of the pharmaceutical composition of the present invention the composition contains less than 1.5% by weight of povidone, preferably less than 1% by weight, more preferably less than 0.75% by weight, more preferably less than 0.50% by weight and even more preferably less than 0.25% by weight calculated with reference to the total weight of the pharmaceutical composition. It has been found that the less the povidone-content of the pharmaceutical composition according to the present invention is, the more is the stability of the pharmaceutical composition with respect to the chemical stability of ruxolitinib.

Pursuant to another preferred embodiment of the pharmaceutical composition of the present invention the composition is free of povidone.

In another preferred embodiment of the pharmaceutical composition of the present invention the composition contains less than 0.50% by weight of a lubricant, preferably less than 0.45% by weight, more preferably less than 0.40% by weight, more preferably less than 0.35% by weight and even more preferably less than 0.30% by weight. Surprisingly it has been found that the less the lubricant-content, in particular the magnesium stearate-content, of the pharmaceutical composition according to the present invention is, the more is the stability of the pharmaceutical composition with respect to the chemical stability of ruxolitinib.

According to another preferred embodiment of the pharmaceutical composition of the present invention said lubricant is magnesium stearate. In particular magnesium stearate adversely effects the chemical stability of ruxolitinib.

Beside ruxolitinib free base or a pharmaceutically acceptable salt thereof the pharmaceutical composition according to the present invention may comprise one or more additional pharmaceutical active ingredients. But, according to another preferred embodiment of the present invention ruxolitinib or the pharmaceutically acceptable salt thereof is the only active pharmaceutical ingredient contained in the composition.

Pursuant to another preferred embodiment of the pharmaceutical composition of the present invention the active pharmaceutical ingredient contained in the composition is ruxolitinib in form of its free base. The ruxolitinib free base is disclosed in EP 1 966 202 A1.

According to another preferred embodiment of the present invention the active pharmaceutical ingredient contained in the composition is ruxolitinib in form of a pharmaceutically acceptable salt. In particular with respect to ruxolitinib salts povidone adversely effects the chemical stability of ruxolitinib.

In accordance with another preferred embodiment of the pharmaceutical composition of the present invention the pharmaceutically acceptable salt of ruxolitinib is selected from the group consisting of a ruxolitinib phosphoric acid salt, a ruxolitinib maleic acid salt, a ruxolitinib sulfuric acid salt and a ruxolitinib oxalic acid salt. A ruxolitinib phosphoric acid salt, a ruxolitinib maleic acid salt and a ruxolitinib sulfuric acid salt is disclosed in EP 2 173 752 A2 in which said salts are described to be a 1:1 salt between ruxolitinib and the acid. A ruxolitinib oxalic acid salt is disclosed in EP 3 183 252 A1.

In another preferred embodiment of the pharmaceutical composition of the present invention the pharmaceutically acceptable salt of ruxolitinib is a ruxolitinib phosphoric acid salt.

The pharmaceutical composition according to the invention can be formulated for immediate, modified, controlled, sustained, prolonged, extended or delayed release of the ruxolitinib drug. However, according to a preferred embodiment of the present invention the pharmaceutical composition is an immediate release composition. Immediate, modified, controlled, sustained, prolonged, extended or delayed release pharmaceutical compositions and their preparation are well known in the prior art and belong the general common knowledge of the skilled person.

The pharmaceutical composition according to the present invention is a solid and thus can be for example in the form of a tablet, capsule, pill, granulate, powder and so on. However, according to a preferred embodiment of the present invention the pharmaceutical composition is a tablet, in particular a filmless tablet.

According to another preferred embodiment of the pharmaceutical composition of the present invention the pharmaceutical composition contains the active pharmaceutical ingredient ruxolitinib or the pharmaceutically acceptable salt thereof-calculated as free base—in the range from 2 mg to 50 mg. In particular the pharmaceutical composition of the present invention comprises 5 mg, 10 mg, 15 mg, 20 mg or 25 mg ruxolitinib free base or the pharmaceutically acceptable salt thereof-calculated as free base.

Pursuant to another preferred embodiment of the pharmaceutical composition of the present invention the pharmaceutical composition is free of acidic reacting pharmaceutical excipients. It has been found that the less the content of the pharmaceutical composition according to the present invention in relation to acidic reacting pharmaceutical excipients is, the more is the stability of the pharmaceutical composition with respect to the chemical stability of ruxolitinib. Acidic reacting pharmaceutical excipients are known in the art and can be determined by measuring of the pH value of a solution or a suspension of the excipient in water, e.g. by measuring the pH value of a solution or suspension of 10 mg of the excipient in 1 ml of water at room temperature.

According to another preferred embodiment of the pharmaceutical composition of the present invention the composition is prepared by means of a process that comprises a wet granulation step, preferably with water as granulation liquid, in which preferably the active pharmaceutical ingredient is granulated, optionally in admixture with at least one pharmaceutically acceptable excipient.

The invented pharmaceutical composition prepared by the use of said process ensures a sufficient dissolution rate of the active pharmaceutical ingredient from the pharmaceutical composition as well as a sufficient mechanical strength of the composition.

With respect to another preferred embodiment of the pharmaceutical composition of the present invention the composition is free of cellulose ester derivatives, nitrogenous polymers and polymers with unsaturated bonds. Said polymers may adversely affect the chemical stability of ruxolitinib as well as its dissolution.

In accordance with another preferred embodiment of the present invention the pharmaceutical composition further comprises a diluent, a disintegrant, a binder and optionally a lubricant and a glidant.

Diluents are fillers which are used to increase the bulk volume of a pharmaceutical composition. By combining a diluent with the active pharmaceutical ingredient, the final product is given adequate weight and size to assist in production and handling.

According to a preferred embodiment of the present invention diluents are used in an amount of from 15% to 95% by weight, preferably from 50% to 90% by weight based on the total weight of the composition. Suitable examples of diluents to be used in accordance with the present invention include microcrystalline cellulose and lactose monohydrate.

The pharmaceutical composition of the present invention may also contain a binder. Binders ensure that a pharmaceutical composition can be formed having the desired or required mechanical strength. Binders which are suitable for use in accordance with the present invention include hydroxypropyl cellulose, hydroxypropyl methylcellulose and sodium carboxyl methylcellulose. In view of the present invention, binders are preferably used in an amount of from 1% to 20% by weight based on the total weight of the composition.

The pharmaceutical composition of the present invention may also contain a disintegrant. Disintegrants are added to a pharmaceutical composition to promote the breakup of the composition into smaller fragments in an aqueous environment, thereby increasing the available surface area and promoting a more rapid release of the active pharmaceutical ingredient.

Suitable examples of disintegrants to be used in accordance with the present invention include crospovidone, sodium starch glycolate, croscarmellose sodium, and mixtures of any of the foregoing. In the frame of the present invention disintegrants preferably are used in an amount of from 1% to 25% by weight based on the total weight of the composition. A preferred disintegrant in the context of the present invention is sodium starch glycolate, in a preferred amount of from 1% to 5% by weight based on the total weight of the composition.

The pharmaceutical composition of the present invention may also contain a lubricant. Lubricants are generally used in order to reduce sliding friction. In particular, to decrease the friction at the interface between the blend to be encapsulated/tableted and dosator of the encapsulation/tableting machine. Suitable lubricants to be used in accordance with the present invention include magnesium stearate, stearic acid, glyceryl behenate, hydrogenated vegetable oil, talc and glycerine fumarate. A preferred lubricant in the frame of the present invention is magnesium stearate. The lubricant is preferably used in an amount of from 0.01% to 0.5% by weight based on the total weight of the composition, more preferably in an amount of from 0.01% to 0.4% by weight.

The pharmaceutical composition of the present invention may also contain a glidant. Glidants enhance product flow by reducing interparticulate friction. A suitable example for a glidant is colloidal silicon dioxide. The glidant is preferably used in an amount of from 0.01% to 5% by weight based on the total weight of the composition, more preferably in an amount of from 0.01% to 3% by weight.

A typical pharmaceutical composition provided by the present invention is a solid oral pharmaceutical composition in form of a tablet, preferably a filmless tablet, wherein the composition is free of povidone and wherein the composition comprises
as an active pharmaceutical ingredient ruxolitinib or a pharmaceutically acceptable salt thereof, preferably a pharmaceutically acceptable salt of ruxolitinib and more preferably a ruxolitinib phosphoric acid salt, preferably in an amount of from 2 to 15% by weight based on the total weight of the composition;
at least one diluent, preferably selected from the group consisting of microcrystalline cellulose and lactose monohydrate, preferably in an amount of from 70 to 95% by weight based on the total weight of the composition;
a disintegrant, preferably sodium starch glycolate, preferably in an amount of from 1 to 10% by weight based on the total weight of the composition;
a binder, preferably hydroxypropyl cellulose, preferably in an amount of from 1 to 10% by weight based on the total weight of the composition;
optionally a lubricant, preferably magnesium stearate, preferably in an amount of from 0.01 to 0.4% by weight based on the total weight of the composition;
optionally a glidant, preferably colloidal silica, preferably in an amount of from 0.01 to 3.0% by weight based on the total weight of the composition.

The present invention relates also to a process for the preparation of a pharmaceutical composition according to the invention comprising the following steps:
providing ruxolitinib or a pharmaceutically acceptable salt thereof as active pharmaceutical ingredient;
mixing the active pharmaceutical ingredient with a pharmaceutically acceptable excipient to obtain a mixture;
wet granulating said mixture to obtain a wet granulate; and
optionally drying said wet granulate.

In a preferred embodiment of the process of the present invention the process comprises the following steps:
providing ruxolitinib or a pharmaceutically acceptable salt thereof as active pharmaceutical ingredient, preferably a pharmaceutically acceptable salt of ruxolitinib and more preferably a ruxolitinib phosphoric acid salt;
mixing the active pharmaceutical ingredient with a diluent, disintegrant and optionally with a binder as pharmaceutically acceptable excipient to obtain a mixture;
wet granulating said mixture with a granulation liquid, preferably water, to obtain a wet granulate, wherein the granulation liquid optionally contains a binder;
drying said wet granulate to obtain a dried wet granulate;
optionally admixing the dried wet granulate at least with a glidant and/or with a lubricant to obtain a granulation mixture; and
compressing the dried wet granulate or the optional granulation mixture into tablets.

### Comparative Example 1

As comparative example the marketed Jakavi® 5 mg, 10 mg, 15 mg and 20 mg tablets have been analysed.

According to the summary of product characteristics of Jakavi® the tablets contain ruxolitinib in form of the phosphate salt as well as microcrystalline cellulose, magnesium stearate, colloidal anhydrous silica, sodium starch glycolate, povidone, hydroxypropyl cellulose and lactose monohydrate as pharmaceutical excipients. The given ruxolitinib amounts of 5 mg, 10 mg, 15 mg and 20 mg of the Jakavi® tablets are calculated based on the ruxolitinib free base.

By analytical methods it has been determined that said marketed Jakavi® 5 mg, 10 mg, 15 mg and 20 mg tablets consist of 4.125% by weight of a ruxolitinib phosphate salt, of 44.66% by weight of microcrystalline cellulose, of 0.5% by weight of magnesium stearate, of 1.0% by weight of colloidal anhydrous silica, of 3.0% by weight of sodium starch glycolate, of 2.0% by weight of povidone, of 2.0% by weight of hydroxypropyl cellulose (low substituted) and of 42.715% by weight of lactose monohydrate.

Moreover, the average hardness of said marketed Jakavi® 5 mg, 10 mg, 15 mg and 20 mg tablets has been determined according to the provision in Eur.Ph. (European Pharmacopoeia, 8^{th} edition, 2.9.8 “hardness of tablets”, p. 412 (German version)). The 5 mg tablets have a circular shape and show an average hardness of 51 N, the 10 mg tablets have a circular shape and show an average hardness of 73 N, the 15 mg tablets have an oval shape and show an average hardness of 102 N and the 20 mg tablets have a capsule shape and show an average hardness of 105 N.

The in-vitro dissolution rate of ruxolitinib of said tablets was analysed by means of the paddle method (apparatus 2) according to the provision in Eur.Ph. (European Pharmacopoeia, 8^{th} edition, 7^{th} supplement, 2.9.3 “dissolution test for solid oral dosage forms”, p. 7367-7375 (German version)) at 50 rpm in 900 ml buffer at 37° C. at pH values of 1.2 (hydrochloric acid solution) 4.5 (acetate sodium acetate buffer) and 6.8 (phosphoric acid buffer).

The results of the dissolution tests are shown in the following tables, wherein the infinity data correspond to stirring at 150 rpm for 15 min after the 45 min time point.

TABLE 1
dissolution mean values of Jakavi ® 5 mg tablets at different pH
values (volume 900 ml; 50 rpm; N = 3 (number of tablets); the
values in brackets are the RSD [%] (relative standard deviation))

| time | pH 1.2 | pH 4.5 | pH 6.8 |
|---|---|---|---|
| [min] | [%] | [%] | [%] |
| 5 | 86(13.0) | 73(0.8) | 56(10.2) |
| 10 | 95(4.8) | 89(1.1) | 78(0.7) |
| 15 | 97(3.1) | 93(1.1) | 85(0.7) |
| 20 | 98(2.6) | 94(1.1) | 88(1.7) |
| 30 | 99(1.5) | 95(1.1) | 90(2.6) |
| 45 | 100(1.0) | 96(1.2) | 91(2.5) |
| infinity | 101(0.6) | 101(0.0) | 100(0.0) |

TABLE 2
dissolution mean values of Jakavi ® 10 mg tablets at different
pH values (volume 900 ml; 50 rpm; N = 3 (number of tablets);
the values in brackets are the RSD [%] (relative standard deviation))

| time | pH 1.2 | pH 4.5 | pH 6.8 |
|---|---|---|---|
| [min] | [%] | [%] | [%] |
| 5 | 77(5.4) | 70(3.6) | 67(3.1) |
| 10 | 87(2.4) | 86(0.0) | 84(6.5) |
| 15 | 90(1.3) | 89(1.1) | 89(7.3) |
| 20 | 92(1.1) | 91(1.7) | 91(7.2) |
| 30 | 95(0.6) | 92(1.7) | 93(6.6) |
| 45 | 97(0.6) | 93(1.6) | 94(6.4) |
| infinity | 102(1.0) | 100(1.2) | 101(1.5) |

TABLE 3
dissolution mean values of Jakavi ® 15 mg tablets at different
pH values (volume 900 ml; 50 rpm; N = 3 (number of tablets);
the values in brackets are the RSD [%] (relative standard deviation))

| time | pH 1.2 | pH 4.5 | pH 6.8 |
|---|---|---|---|
| [min] | [%] | [%] | [%] |
| 5 | 85(5.4) | 74(6.1) | 66(4.0) |
| 10 | 92(1.7) | 86(4.4) | 82(2.5) |
| 15 | 94(0.6) | 88(3.6) | 86(2.3) |
| 20 | 95(0.6) | 89(3.6) | 88(1.7) |
| 30 | 97(0.6) | 90(3.6) | 90(1.1) |
| 45 | 98(0.6) | 91(2.9) | 91(1.7) |
| infinity | 101(1.0) | 98(0.6) | 99(0.6) |

TABLE 4
dissolution mean values of Jakavi ® 20 mg tablets at different
pH values (volume 900 ml; 50 rpm; N = 3 (number of tablets);
the values in brackets are the RSD [%] (relative standard deviation)

| time | pH 1.2 | pH 4.5 | pH 6.8 |
|---|---|---|---|
| [min] | [%] | [%] | [%] |
| 5 | 83(2.5) | 70(3.0) | 59(2.6) |
| 10 | 90(1.9) | 81(2.6) | 77(3.9) |
| 15 | 92(1.9) | 83(3.0) | 82(4.3) |
| 20 | 93(2.2) | 85(2.5) | 85(4.1) |
| 30 | 94(2.2) | 86(2.4) | 86(4.7) |
| 45 | 96(2.6) | 87(2.4) | 88(4.5) |
| infinity | 100(1.5) | 96(1.0) | 98(0.0) |

TABLE 5
dissolution mean values of Jakavi® 20 mg + 5 mg tablets
(one Jakavi ® 20 mg tablet and one Jakavi ® 5 mg tablet)
at different pH values (volume 900 ml; 50 rpm;
N = 3 (number of tablet pairs); the values in brackets
are the RSD [%] (relative standard deviation))

| time | pH 1.2 | pH 4.5 | pH 6.8 |
|---|---|---|---|
| [min] | [%] | [%] | [%] |
| 5 | 85(9.3) | 59(3.5) | 58(4.3) |
| 10 | 91(7.3) | 72(2.1) | 72(2.1) |
| 15 | 93(6.2) | 76(2.0) | 76(2.0) |
| 20 | 95(5.8) | 78(1.5) | 77(2.0) |
| 30 | 96(5.1) | 80(2.2) | 80(2.6) |
| 45 | 98(4.8) | 82(2.5) | 82(2.4) |
| infinity | 104(2.8) | 92(1.3) | 92(1.1) |

Moreover, the stability of Jakavi® 20 mg tablets was investigated in view of the chemical stability of ruxolitinib, namely the original value directly after purchase of the tablets as well after storage in aluminium-aluminium packages under accelerated conditions at 40° C./75% relative humidity/15 days or at 60° C./75% relative humidity/15 days. The results of the stability study are shown in the following Table 6.

TABLE 6
Stability of Jakavi ® 20 mg tablets

| storage conditions | total impurities/% |
|---|---|
| original | 0.2 |
| 40° C./75% RH/15 d | 0.22 |
| 60° C./75% RH/15 d | 0.62 |

The data in Table 6 have been determined by means of high performance liquid chromatography and evaluated by using the area normalization method. The high performance liquid chromatography was conducted with the following parameters:

High performance liquid chromatography, chromatographic conditions:
column: Zorbax Eclipse XDB C18, 3.5 μm (150×4.6 mm);
mobile phases: A water, B acetonitrile, linear elution (below)

| Time(min) | A(%) | B(%) |
|---|---|---|
| 0.0 | 85 | 15 |
| 4.0 | 70 | 30 |
| 12.0 | 50 | 50 |
| 25.0 | 10 | 90 |
| 25.1 | 85 | 15 |
| 30.0 | 85 | 15 |

detection wavelength: 220 nm;
velocity of flow: 1.0 ml/min
column temperature: 30° C.;
test solvent: acetonitrile-water (1:1);
test concentration: 250 μg/ml.

### Example 1

Ruxolitinib phosphate salt tablets—containing 5 mg, 10 mg, 15 mg and 20 mg (calculated as ruxolitinib free base) ruxolitinib phosphate salt—with the following constitution have been prepared by wet granulation techniques:

| | tablet [% by |
|---|---|
| ingredient | weight] |
| ruxolitinib phosphate (API) | 4.1 |
| microcrystalline cellulose (PH101) | 40.6 |
| lactose monohydrate | 47.5 |
| sodium starch glycolate (type A) | 3.1 |
| hydroxypropyl cellulose (HPC-L, l | 3.1 |
| ow-substituted) | |
| colloidal silica | 1.2 |
| magnesium stearate | 0.4 |
| water for 1^{st} granulation step | (16.25, not contained in the |
| | final product) |
| water for 2^{nd} granulation step | (16.25, not contained in the |
| | final product) |
| total | 100 |
| process | two-step |

In a first stage of the wet granulation step ruxolitinib phosphate and microcrystalline cellulose are (dry) mixed for 10 minutes (stir at 150 RPM, knife 2000 RPM). Then water (water for the 1^{st} granulation step) is added and the mixture granulated for 5 minutes.

In a second stage of the wet granulation step the excipients HPC-L, lactose monohydrate and sodium starch glycolate are added to said wetted granulation mixture and the resulting mixture is stirred and mixed (stir at 150 RPM, knife 2000 RPM) for 10 minutes. The remaining water (water for the 2^{nd} granulation step) is added and the granulation is continued for 5 minutes.

The obtained granules are wet milled, sieved (mesh 1.5 mm, gasket 4.0 mm, 800 rpm) and the obtained granules are dried at 40° C. to a LOD of 2.3%.

The dried granules are mixed with colloidal silica, sieved, lubricated with magnesium stearate and pressed into tablets.

The 5 mg tablets have a circular shape and show an average hardness of 61 N, the 10 mg tablets a circular shape and an average hardness of 95 N, the 15 mg tablets an oval shape and an average hardness of 99 N and the 20 mg tablets a capsule shape and an average hardness of 141 N. The tablet hardness is determined as described above in the chapter “Comparative example 1”.

The results of dissolution tests-performed as described in chapter “Comparative example 1”—of tablets of example 1 are shown in the following tables, wherein the infinity data correspond to stirring at 150 rpm for 15 min after the 45 min time point.

TABLE 7
dissolution mean values of 5 mg tablets at different pH values
(volume 900 ml; 50 rpm; N = 3 (number of tablets); the values
in brackets are the RSD [%] (relative standard deviation))

| time | pH 1.2 | pH 4.5 | pH 6.8 |
|---|---|---|---|
| [min] | [%] | [%] | [%] |
| 5 | 92(7.6) | 55(6.3) | 52(3.8) |
| 10 | 99(3.1) | 76(5.0) | 77(3.9) |
| 15 | 101(2.3) | 86(3.5) | 88(2.6) |
| 20 | 102(2.3) | 92(2.7) | 94(1.8) |
| 30 | 102(2.0) | 97(2.6) | 99(0.6) |
| 45 | 103(1.9) | 100(2.5) | 102(0.6) |
| infinity | 103(1.9) | 103(1.1) | 104(0.6) |

TABLE 8
dissolution mean values of 20 mg tablets at different pH
values (volume 900 ml; 50 rpm; N = 3 (number of tablets); the values
in brackets are the RSD [%] (relative standard deviation))

| time | pH 1.2 | pH 4.5 | pH 6.8 |
|---|---|---|---|
| [min] | [%] | [%] | [%] |
| 5 | 77(2.0) | 65(1.3) | 63(3.1) |
| 10 | 88(2.0) | 77(0.9) | 81(2.2) |
| 15 | 91(2.3) | 79(1.5) | 88(1.7) |
| 20 | 91(2.3) | 82(1.4) | 91(1.5) |
| 30 | 94(2.2) | 86(1.8) | 93(1.5) |
| 45 | 95(1.6) | 89(1.7) | 95(1.2) |
| infinity | 100(0.6) | 100(2.1) | 98(0.8) |

TABLE 9
dissolution mean values of 25 mg tablets at different pH values
(volume 900 ml; 50 rpm; N = 3 (number of tablet pairs); the
values in brackets are the RSD [%] (relative standard deviation))

| time | pH 1.2 | pH 4.5 | pH 6.8 |
|---|---|---|---|
| [min] | [%] | [%] | [%] |
| 5 | 74(5.4) | 51(1.1) | 43(2.3) |
| 10 | 85(3.5) | 69(1.4) | 62(0.9) |
| 15 | 88(3.0) | 77(1.5) | 70(2.2) |
| 20 | 90(2.3) | 80(1.9) | 75(2.8) |
| 30 | 92(2.3) | 84(2.4) | 79(3.3) |
| 45 | 93(1.6) | 86(2.3) | 82(2.5) |
| infinity | 100(1.0) | 100(1.0) | 101(0.6) |

Moreover, the stability of the tablets according to the invention was investigated in view of the chemical stability of ruxolitinib, namely the original value directly after preparation of the tablets as well after storage in aluminium-aluminium packages under accelerated conditions at 40° C./75% relative humidity/15 days or at 60° C./75% relative humidity/15 days. The results of the stability study are shown in the following Table 10:

TABLE 10
Stability of Ruxolitinib phosphate 20 mg tablets

| | total impurities |
|---|---|
| storage condition | [%] |
| original | 0.10 |
| 40° C./75% RH/15 d | 0.10 |
| 60° C./75% RH/15 d | 0.20 |

The data in Table 10 have been determined and evaluated with methods as described in the above chapter “Comparative example 1”.

A comparison of the stability data of the comparative example 1 with the stability data of example 1 shows clearly that the stability of ruxolitinib is significantly higher in the composition according to the present invention. This comparison is also shown in Table 11 below.

TABLE 11
comparison of stability data

| | Jakavi ® | Ruxolitinib |
|---|---|---|
| | 20 mg | phosphate |
| | tablets | 20 mg tablets |
| storage condition | total impurities [%] | total impurities [%] |
| original | 0.2 | 0.10 |
| 40° C./75% RH/15 d | 0.22 | 0.10 |
| 60° C./75% RH/15 d | 0.62 | 0.20 |

Furthermore, a comparison of the dissolution data of the comparative example 1 with the dissolution data of the present example according to the present invention reveals that the dissolution rates are comparable.

1. A solid oral pharmaceutical composition comprising as an active pharmaceutical ingredient ruxolitinib or a pharmaceutically acceptable salt thereof, wherein the composition contains less than 2.0% by weight of povidone.

2. The pharmaceutical composition according to claim 1, wherein the composition contains less than 1.5% by weight of povidone.

3. The pharmaceutical composition according to claim 1, wherein the pharmaceutical composition is free of povidone.

4. The pharmaceutical composition according to claim 1, wherein the composition contains less than 0.50% by weight of a lubricant.

5. The pharmaceutical composition according to claim 4, wherein the lubricant is magnesium stearate.

6. The pharmaceutical composition according to claim 1, wherein the active pharmaceutical ingredient ruxolitinib or the pharmaceutically acceptable salt thereof is the only active pharmaceutical ingredient contained in the composition.

7. The pharmaceutical composition according to claim 1, wherein the active pharmaceutical ingredient contained in the composition is ruxolitinib in form of its free base.

8. The pharmaceutical composition according to claim 1, wherein the active pharmaceutical ingredient contained in the composition is ruxolitinib in form of a pharmaceutically acceptable salt.

9. The pharmaceutical composition according to claim 8, wherein the pharmaceutically acceptable salt of ruxolitinib is selected from the group consisting of a ruxolitinib phosphoric acid salt, a ruxolitinib maleic acid salt, a ruxolitinib sulfuric acid salt-salt, and a ruxolitinib oxalic acid salt.

10. The pharmaceutical composition according to claim 9, wherein the pharmaceutically acceptable salt of ruxolitinib is the ruxolitinib phosphoric acid salt.

11. The pharmaceutical composition according to claim 1, wherein the composition is an immediate release composition.

12. The pharmaceutical composition according to claim 1, wherein the pharmaceutical composition is a tablet.

13. The pharmaceutical composition according to claim 1, wherein the composition comprises the active pharmaceutical ingredient ruxolitinib or the pharmaceutically acceptable salt thereof—calculated as free base—in a range from 2 mg to 50 mg.

14. The pharmaceutical composition according to claim 1, wherein the composition comprises 5 mg, 10 mg, 15 mg, 20 mg or 25 mg ruxolitinib or the pharmaceutically acceptable salt thereof—calculated as free base.

15. The pharmaceutical composition according to claim 1, wherein the pharmaceutical composition is free of acidic reacting pharmaceutical excipients.

16. The pharmaceutical composition according to claim 1, wherein the composition is prepared by a process that comprises a wet granulation step, optionally in admixture with at least one pharmaceutically acceptable excipient.

17. The pharmaceutical composition according to claim 1, wherein the composition further comprises a diluent, a disintegrant, a binder and optionally a lubricant and/or a glidant.

18. A process for the preparation of a pharmaceutical composition according to claim 1, the process comprising the following steps:
a) providing ruxolitinib or the pharmaceutically acceptable salt thereof as the active pharmaceutical ingredient;
b) mixing the active pharmaceutical ingredient with a pharmaceutically acceptable excipient to obtain a mixture;
c) wet granulating the mixture to obtain a wet granulate; and
d) optionally, drying the wet granulate.

19. The pharmaceutical composition according to claim 16, wherein the active pharmaceutical ingredient is granulated.
